(19) United States
(12) Patent Application Publication
(10) Pub. No.: US 2017/0189902 A1
(43) Pub. Date: Jul. 6, 2017

(54) READY-TO-CONSTITUTE ANALYTICAL PLATFORMS FOR CHEMICAL ANALYSES AND QUANTIFICATION

(71) Applicant: PinPoint Testing, LLC, Little Rock, AR (US)

(72) Inventor: Jeffery H. Moran, Little Rock, AR (US)

(73) Assignee: PinPoint Testing, LLC, Little Rock, AR (US)

(21) Appl. No.: 15/510,633

(22) PCT Filed: Sep. 11, 2015
(86) PCT No.: PCT/US15/49721

§ 371 Date: Mar. 10, 2017

Related U.S. Application Data
(60) Provisional application No. 62/049,756, filed on Sep. 12, 2014.
(60) Provisional application No. 62/105,478, filed on Jan. 20, 2015.

(51) Int. Cl. B01L 3/00; G01N 33/49; G01N 33/493; G01N 33/94; G01N 33/96

(52) U.S. Cl. CPC B01L 3/5085; G01N 33/946; G01N 33/96; G01N 33/9486; G01N 33/94; G01N 33/493; G01N 33/492; B01L 2200/148; B01L 2200/16; B01L 2300/0829; G01N 2458/15; B01L 2200/141

(57) ABSTRACT

The present invention provides devices, kits and methods for the rapid multiplex quantitative analysis of analytes in a sample while eliminating the need for the end user to prepare standardized solutions of the analytes or internal standards. The devices of the present invention comprise multiwell plates manufactured to contain dried calibration standards, dried quality control standards, and dried internal standards and optionally contain tracers and deconjugation enzymes. The methods of the present invention do not require preparation and addition of these standards or optional components to a device, thus eliminating steps costly with regard to time and sample analyte measurement precision.

### CROSS REFERENCE TO RELATED APPLICATIONS

This PCT application claims the benefit of U.S. Provisional Application Ser. No. 62/049,756, filed Sep. 12, 2014, and U.S. Provisional Application Ser. No. 62/106,478, filed Jan. 22, 2015. The disclosures of these documents are hereby incorporated by reference in their entireties.

### FIELD OF THE INVENTION

The present invention relates to devices, kits, and methods for the quantitative analysis of an analyte or analytes in a sample.

### BACKGROUND

Multiplex quantitative analysis of analytes in a test sample typically requires the incorporation of internal standards into the mixture to be analyzed and detection methods of suitable sensitivity and precision for generating valid measurements. Assays designed and produced to carry out such quantitative analyses using antibody or mass spectrometry detection techniques, for example, are well known. Quantitative assays known in the art require preparation and addition of internal and quality control standard solutions at the point of use that may add costs, time, and uncertainty to measurements. In addition, depending on the application, with the prior art assays the end user may need to identify and obtain specific certified reference material adding one more step that may require significant time at the point of use to ensure that the required standards are met. Several robotic systems are also available to assist analyst in streamlining sample preparation and processing protocols. Cross-contamination (e.g. well-to-well contamination) is always a concern and must be adequately controlled in laboratories to guard data quality. This is a challenging process that is prone to error. This can be even more challenging when laboratories rely heavily on robotics and analyst are not present to observe every step of the sample preparation process. Thus, there is a need for methods and devices that are more efficient, rapid, sensitive, reliable, cost-effective, and easy-to-use and for analytical tools to assist laboratories in adequately controlling for cross-contamination. The present invention addresses these issues by providing high and medium throughput multiplex quantitative assays of high precision and accuracy to meet the growing demand of clinicians, toxicologists, researchers, and environmental, food, and forensic scientists.

### SUMMARY

The present invention provides devices for quantifying the concentration of a plurality of analytes in a test sample, wherein the device comprises a multi-well plate wherein each well independently is left empty or independently comprises a dried calibration standard, a dried quality control standard, a dried internal standard, or any combination thereof; or a plurality of vials wherein each vial is left empty or independently comprises a calibration standard, a quality control standard, an internal standard, or any combination thereof.

The present invention additionally provides devices for quantifying the concentration of a plurality of analytes in a test sample, wherein the device comprises a multi-well plate where each well independently is left empty or independently comprises a dried calibration standard, a dried quality control standard, a dried internal standard, or any combination thereof; and further comprises a tracer to allow for detection of cross-contamination between wells of the device.

The present invention also provides kits for quantitative determination of the concentration of a plurality of analytes in one or a plurality of test samples comprising a device according of the invention and a detailed written description of the specifications of the device.

The present invention also provides for methods of determining the concentration of a plurality of analytes in one or a plurality of test samples using a device of the present invention comprising the steps of: i) providing a device of the present invention wherein the device is a multiwell plate comprising a plurality of wells containing a plurality of calibration standards and a plurality of internal standards (CS+IS), a plurality of wells containing a plurality of quality control standards and a plurality of internal standards (QC+IS), and a plurality of wells containing a plurality of internal standards (IS); ii) adding a blank sample into each of the wells containing the CS+IS and into each of the wells containing the QC+IS; iii) adding a test sample into a well containing IS; iv) mixing the well contents to generate a plurality of well samples; v) preparing the well samples for quantitative analysis; vi) quantitative analysis of the well samples; and vii) determining the concentration of the analytes present in the test samples, wherein steps ii) and iii) can be performed in any order.

### BRIEF DESCRIPTION OF THE FIGURES

FIG. 1 illustrates the percent recovery of analytes from a device of the invention after quantification of a test sample from blood (FIG. 1A) and urine (FIG. 1B).

FIG. 2 illustrates a line graph depicting a comparison of the efficacy of β-glucuronidase pre-manufactured in a 96-well plate in accordance with the device and methods of the present invention with the efficacy of β-glucuronidase added using traditional liquid methods. Morphine formation from morphine-3β-D-glucuronide spiked in urine was used as a marker of deconjugation rate and measured using LC-ToF-mass spectrometery.

FIG. 3 illustrates a standard curve of

### DETAILED DESCRIPTION

### Definitions

A “reference standard,” as used herein is a standardized analyte which is used as a measurement base for the analyte to be tested.

An “internal standard” and “IS” are used interchangeably herein and refer to a reference standard that is modified for detection or is a surrogate reference standard labeled for detection. For example, the internal standard may be a reference standard having at least one atomic substitution in its molecular structure.

A “surrogate reference standard” is a substitute for a reference standard. For example the racemic analyte (+/−)-methamphetamine-d3 may be used as a surrogate standard for the chiral analyte (+)-methamphetamine-d3.

A “calibration standard” and “CS” are used interchangeably herein and refer to a reference standard that is used to calibrate an instrument reading with an amount of an analyte.

“Second-source quality control standard”, “quality control standard” and “QC” as used interchangeably herein, and refer to a reference standard that is 1) obtained or prepared from a source independent of the source of the calibration standard, or 2) is obtained or prepared from a reference standard from the same source as the calibration standard but from a different lot than the reference standard used to prepare the calibration standard, or 3) the quality control standard is made from the same source and lot but from independent preparation. The second-source quality control standard is used to verify the correctness of a calibration obtained using the calibration standard.

“Blank matrix” or “biological matrix” means a sample similar to the batch of associated test samples that is free from the analytes of interest and is processed simultaneously with and under the same conditions as samples through all steps of the analytical procedures, and in which no target analytes or interferences are present at concentrations that significantly impact the analytical results for sample analyses. For example, where the sample to be tested is urine, the blank matrix or biological matrix would be urine that does not contain any of the analytes of interest.

When referring to a well or vial as “blank” it means that the well or vial does not contain any internal standard, quality control standard, calibration standard or the like.

Where a well or vial of the device of the present invention is described as “containing” a CS, an IS, or a QC it is to be interpreted as excluding the unrecited standards. For example, where a well is described as containing a CS and an IS, the well does not contain a QC. Similarly, if a well is described as containing an IS, the well does not contain a CS or an QC. If a well is described as containing a QC and an IS, the well does not contain a CS.

“Manufactured to contain” means that the internal standard, quality control standard, calibration standard, or the like, are added to the wells or vials of the device and dried prior to receipt of the device by the end user.

6-MAM is an abbreviation for 6-monoacetylmorphine.

EDDP is an abbreviation for 2-ethylidene-1,5-dimethyl-3-diphenylpyrrolidine.

MDA is an abbreviation for 3,4-methylenedioxyamphetamine.

MDEA is an abbreviation for 3,4-methylenedioxyethylamphetamine.

MDMA is an abbreviation for 3,4-methylenedioxymethamphetamine.

“A,” “an,” “the,” “at least one,” and “one or more” are used interchangeably to indicate that at least one of the items is present; a plurality of such items may be present unless the context clearly indicates otherwise. As used herein a plurality of items can mean two or more of these items. A plurality of wells can mean two or more wells, or even all of the wells of the device.

It is noted that in this disclosure, terms such as “comprises”, “comprised”, “comprising”, “contains”, “containing” and the like have the meaning attributed in United States Patent law; they are inclusive or open-ended and do not exclude additional, un-recited elements or method steps unless clearly specified otherwise in the present invention. Terms such as “consisting essentially of” and “consists essentially of” have the meaning attributed in United States Patent law; they allow for the inclusion of additional ingredients or steps that do not materially affect the basic and novel characteristics of the claimed invention. The terms “consists of” and “consisting of” have the meaning ascribed to them in United States Patent law; namely that these terms are close ended.

The antecedent “about” indicates that the values are approximate. For example the range of “about 1 mg to about 50 mg” indicates that the values are approximate values. The range of “about 1 mg to about 50 mg” includes approximate and specific values, e.g., the range includes about 1 mg, 1 mg, about 50 mg and 50 mg.

When a range is described, the range includes both the endpoints of the range as well as all numbers in between. For example, “between 1 mg and 10 mg” includes 1 mg, 10 mg and all amounts between 1 mg and 10 mg, for example, 0.1 mg, 2 mg, 5.6 mg, 9.75 mg, and 9.9 mg. Likewise, “from 1 mg to 10 mg” includes 1 mg, 10 mg and all amounts between 1 mg and 10 mg, for example, 0.1 mg, 2 mg, 5.6 mg, 9.75 mg, and 9.9 mg.

The present disclosure provides ready-to-use assay kits and methods for the rapid multiplex quantitative analysis of analytes in a test sample while eliminating the need for the end user to prepare standardized solutions of the analytes, calibration standards, second-source quality control standards, or internal standards.

Another aspect of the invention includes the creation and use of differentiated internal standards for isobaric analytes and analytes that may degrade to other analytes of interest. For example hydromorphone and morphine are isobaric (are distinct compounds of the same mass). By the incorporation of differing levels of deuterium in the internal standards, the resulting differing masses of the internal standards of these two compounds will simplify their analysis and eliminate the potential for misidentification. Hydrocodone and codeine are two additional applicable isobaric examples. Another example where differentiation through varied incorporation of deuterium offers an advantage is when there are degradation products that are also analytes of interest. For example, heroin may degrade to 6-MAM which may be further degraded to morphine. Each of these three compounds are common analytes of interest. By the incorporation of differing level of deuterium, the internal standards used on the plate for these three items will be distinct from the degradive products (i.e. 6-MAM form through the degradation of heroin) which will eliminate the potential for misidentification or the potential for inaccurate quantification.

Devices

One aspect of the present invention provides a device that comprises a multiwell assay plate comprising a certain number of wells wherein the device is manufactured such that each well independently contains a precise, pre-determined quantity of a calibration standard, a precise, pre-determined quantity of a quality control standard, a precise, pre-determined quantity of an internal standard, or is left blank. Another aspect the present invention provides a device that comprises a multiwell assay plate comprising a certain number of wells wherein the device is manufactured such that each well independently contains one or more deconjugation enzymes, a precise, pre-determined quantity of a calibration standard, a precise, pre-determined quantity of a quality control standard, a precise, pre-determined quantity of an internal standard, or is left blank. Another aspect of the present invention provides a device that comprises chemical tracers that enable the detection of cross-contamination that may occur during use.

In one embodiment, the multiwell assay plate is a 48-well plate. In another embodiment the multiwell plate is a 96-well plate. In yet another embodiment, the multiwell plate is a 384-well plate. In still another embodiment the multiwell plate is a 1536 well plate. In various embodiments, the multiwell assay plate can be manufactured using any chemically compatible plastics and solid substrates. In some embodiments, the multiwell assay plate is suitable for in-situ fluorescence or chemillminescence analysis. In various embodiments, the multiwell assay plate is biologically inert, non-toxic, compatible with various aqueous and organic solvents, does not leach any chemical residues, and does not interfere with the quantitative analysis of the well samples.

For certain analyses the volume of the test sample is greater than the volume of a well of a multiwell plate. Therefore, another aspect of the invention provides for a device that comprises containers such as vials or tubes which are used in place of the wells of a multiwell plate. In some embodiments of the invention the containers are supported by a vial tray. In one embodiment the vial tray is a 54-position vial tray.

It should be understood that the contents of each of the wells or vials in a device are independent of each other such that a percentage of the wells or vials may contain only an internal standard, a percentage of the wells or vials may contain a calibration standard and an internal standard, a percentage of wells may be empty, etc., depending upon the specific assay to be performed. Further, it should be understood that although an embodiment is described in terms of a multiwell plate device, unless clearly stated otherwise, the same components and configurations as described for multiwell plate can be used where the device is a vial tray or other type of container, scaling up as appropriate.

In one embodiment of the invention, the device is a multiwell plate comprising wells that contain within each well one or more calibration standards and one or more internal standards (CS+IS), wells that contain a plurality of internal standard only (IS), wells that contain a quality control standard and an internal standard (QC+IS), and wells that are empty (blank). In yet another embodiment the device is a vial tray comprising vials that contain a calibration standard and an internal standard (CS+IS), vials that contain an internal standard only (IS), vials that contain a quality control standard and an internal standard QC+IS), and vials that are empty (blank).

After addition of the desired components, the plates or vials are dried. The plates can be dried after addition of one, or two, or all of the components. The components can be added in any order or simultaneously. In some embodiments of the device a solvent may be added to one or more wells after the other components are dried. Methods for drying are well known in the art and include, but are not limited to, lyophilization, evaporation under normal or reduced atmosphere, or under stream of inert gas such as nitrogen or argon. In some embodiments, the device can be dried after the addition of one, or two, or all of the components using mild heating conditions, e.g. from about 21° C. to about 75° C., or from about 30° C. to about 50° C., with or without reduced or negative pressure, for example in a vacuum. In some embodiments, the device can be dried after the addition of one, or two, or all of the components at room temperature between atmospheric pressure and 10⁻⁵ atmospheres. The mild heating of the devices to dry any residual solvents may proceed for a period of 30 minutes to 48 hours. In some embodiments, the degree of drying can be measured by determining the residual volume of fluid left in each of the wells. In some embodiments, the wells may be dried to remove residual solvent (aqueous or organic) and be left with a residual solvent ranging from 0% to about 1% (v/v), or from about 0.001% to about 0.99% (v/v), or from about 0.01% to about 0.9% (v/v), or from about 0.1% to about 0.9% (v/v).

In some embodiments, the analyte(s) to be quantified in a test sample are drugs, including but not limited to, synthetic drugs, natural drugs, prescription drugs and over the counter drugs. The following are nonlimiting examples of such drugs that may be quantitated using the devices of the present invention: norpropoxyphene, propoxyphene, amphetamine, 3,4-methylenedioxyamphetamine (MDA), 3,4-methylenedioxy-N-ethylamphetamine (3,4-MDEA), 3,4-methylenedioxy-N-methylamphetamine (3,4-MDMA), methadone, methamphetamine, 6-monoacetylmorphine (6-MAM), 7-aminoclonazepam, α-hydroxyalprazolam, acetyl fentanyl, acetyl norfentanyl, alprazolam, benzoylecgonine, buprenorphine, carisoprodol, clonazepam, cocaine, codeine, cyclobenzaprine, dextromethorphan, diazepam, dihydrocodeine, 2-ethylidene-1,5-dimethyl-3,3-diphenylpyrrolidine (EDDP), fentanyl, flunitrazepam, flurazepam, heroin, hydrocodone, hydrodone, hydromorphone, lorazepam, meperidine, meprobamate, midazolam, morphine, naloxone, naltrexone, nitrazepam, norbuprenorphine, nordiazepam, norfentanyl, normeperidine, oxazepam, oxycodone, oxymorphone, phenazepam, phencyclidine (PCP), phentermine, sufentanil, temazepam, cis-tramadol, ecgonine methyl ester, anhydroecgonine methyl ester, cocaethylene, 6-acetylcodeine, N-methyl-1,3-benzodioxolylbutanamine (MBDB), mephedrone, normephodrone, cathinone, Δ9-tetrahydrocannabinol (Δ9-THC), tetrahydrocannabinolic acid, ketamine, gabapentin, pregabalin, and norsufentanil.

Therefore, in some embodiments of the disclosed device, the device comprises internal standards, quality control samples, calibration standards which comprise a plurality of drugs selected from the group consisting of norpropoxyphene, propoxyphene, amphetamine, 3,4-methylenedioxyamphetamine (MDA), 3,4-methylenedioxy-N-ethylamphetamine (3,4-MDEA), 3,4-methylenedioxy-N-methylamphetamine (3,4-MDMA), methadone, methamphetamine, 6-monoacetylmorphine (6-MAM), 7-aminoclonazepam, α-hydroxyalprazolam, acetyl fentanyl, acetyl norfentanyl, alprazolam, benzoylecgonine, buprenorphine, carisoprodol, clonazepam, cocaine, codeine, cyclobenzaprine, dextromethorphan, diazepam, dihydrocodeine, 2-ethylidene-1,5-dimethyl-3,3-diphenylpyrrolidine (EDDP), fentanyl, flunitrazepam, flurazepam, heroin, hydrocodone, hydrodone, hydromorphone, lorazepam, meperidine, meprobamate, midazolam, morphine, naloxone, naltrexone, nitrazepam, norbuprenorphine, nordiazepam, norfentanyl, normeperidine, oxazepam, oxycodone, oxymorphone, phenazepam, phencyclidine (PCP), phentermine, sufentanil, temazepam, cis-tramadol, ecgonine methyl ester, anhydroecgonine methyl ester, cocaethylene, 6-acetylcodeine, N-methyl-1,3-benzodioxolylbutanamine (MBDB), mephedrone, normephodrone, cathinone, Δ9-tetrahydrocannabinol (Δ9-THC), tetrahydrocannabinolic acid, ketamine, gabapentin, pregabalin, and norsufentanil.

In some embodiments, the analyte to be quantified in a test sample is an environmental pollutant such as aromatic hydrocarbons for example benzene or naphthalene, pesticides, herbicides, toxins, hormones and the like.

In some embodiments the internal standard is reference standard wherein the reference standard has at least one atomic substitution in its molecular structure.

In some embodiments, the atomic substitution is an isotope of the substituted atom. In some embodiments, the substituted atom is hydrogen and the isotope is deuterium. In some embodiments, the substituted atom is carbon-12 and the isotope is carbon-13. In some embodiments, the substituted atom is nitrogen-14 and the isotope is nitrogen-15. In some embodiments, the substituted atom is oxygen-16 and the isotope is oxygen-18.

In some embodiments, the atomic substitution is a hydrogen atom substituted with atom that is not carbon or nitrogen. In some embodiments, the other atom is fluorine.

The devices of the present disclosure may be configured to quantify a panel of analytes. For example, the device may be configured to detect benzodiazepines. Thus, in one embodiment the plate is configured to detect one or more analytes including: 7-aminoclonazepam, alprazolam, clonazepam, diazepam, flunitrazepam, flurazepam, α-hydroxalprazolam, lorazepam, midazolam, nitrazepam, nordiazepam, oxazepam, phenazepam and temazepam. Or in other embodiments the device may be configured for the quantification of specific drugs of abuse, for example narcotics. Thus in another embodiment the device is configured to quantify analytes selected from the group consisting of amphetamine, benzolylecgonine, carisoprodol, cocaine, clyclobenzaprine, MDA, MDEA, MDMA, methamphetamine phencyclidine and phentermine.

The various embodiments of the devices of the present disclosure can be further customized to comprise one or more deconjugation enzymes. Many drugs undergo metabolism in the body prior to being excreted. Drug metabolism is generally the physiological modification of pharmaceutical substances to more water soluble derivatives, e.g., though specialized enzymatic systems, to facilitate detoxification and excretion in bile and/or urine. Drug metabolism can be divided into phases, Phase 1 generally involves oxidation, reduction and/or hydrolysis. Phase II involves conjugation of the drug to a charged molecule such as glutathione (GSH), sulfate, glycine or gluronic acid. Products of conjugation have increased molecular weight compared with the parent drug and the attachment of an ionized group makes the metabolite more water soluble. These conjugation reactions are catalyzed by a large group of broad-specificity transferases, including but not limited to, methyltransferase, sulfotransferases, n-acetyltransferases, UDP-glucuronosyltranferases, glutathione S-transferases, and acetyl Co-enzyme As, which are responsible for methylation, sulphation, acetylation, glucuronidation, glutathione conjugation or glycine conjugation, respectively.

There are also enzymes that catalyze the degradation or hydrolysis of the conjugate, i.e. they deconjugate the drug from the drug-conjugate. For example, β-glucuronidase, sulfatases, and glutathione sulfatase catalyze the hydrolysis of glucuronic acid, sulfate, and glutathione conjugates respectively. As used herein, the term “deconjugation enzyme” encompasses enzymes that catalyze deconjugation of a drug-conjugate.

Conjugation of the drug may prevent, or interfere with its detection in a patient sample by a method such as mass spectroscopy. This problem has been previously addressed by pretreating the sample with one or more deconjugation enzymes in a step prior to the addition of the sample to an assay device.

The present disclosure provides devices which incorporates a deconjugation enzyme built into the device and avoids possible errors and inconsistencies in enzyme deconjugation during sample preparation. In one such embodiment of a device of the invention, the device is a multiwell plate comprising wells that contain a plurality of calibration standard and a plurality of internal standard (CS+IS), wells that contain a plurality of internal standard only (IS), wells that contain a quality control standard and an internal standard (QC+IS), wells that contain one or a plurality of deconjugation enzymes, and wells that are empty (blank). In one embodiment of the present invention, the device is manufactured to contain β-glucuronidase in a plurality of the wells of the device. The amount of β-glucuronidase per well may vary depending for example, on the type, amount, and/or concentration of sample that will be added to the plate by the end user. The amount of β-glucuronidase can be varied in different wells or ascertained emipirically for specific drg-conjugatte species. Although β-glucuronidase is exemplified, the same consideration applies to the other deconjugation enzymes. In a particular embodiment the sample is a urine sample and the wells of the device comprise β-glucuronidase. In another embodiment, the device is manufactured to contain a sulphatase.

Another embodiment of the present invention is a device that incorporates tracers that allow for detection of cross-contamination between the wells of a multiwell plate that may occur during performance of an assay. A tracer can be, for example, a uniquely labeled standard or a standard or other reagent that is not the same and another component in the assays and does not interfere any of the components in the assay or interfere with the detection of the analytes. The tracer must also effectively track along with the analytes of interest, for example, a tracker must have the same or similar extraction efficiency of the analytes of interest. Particularly suitable trackers are the same compound as an internal standard but differentially labeled. For example if a morphine-d6 derivative is used as an internal standard, a differentially labeled morphine derivative such as ¹³C-labeled morphine-derivative, a morphine-d7 derivative, a ¹³C-labeled morphine-d6 derivative and a ¹³C labeled morphine-d7 derivative would be suitable tracers. In one embodiment of the device of the present invention is a 96-well plate (e.g. a 12 column×8 row 96-well plate) comprising four different tracers which are configured in the wells as follows: the first tracer is added to every other row consisting of 12 individual wells. The second tracer is added to the rows that were previously skipped. Similarly the third tracer is added to every other column consisting of 8 individual wells. The fourth tracer is added to the columns that were previously skipped. One of skill in the art based on the present disclosure could construct other tracer configurations for use in wells of a multi-well plate. For example, some configurations may allow only for detection of vertical contamination. Other configurations may allow only for detection of horizontal contamination. And yet other configurations would allow for detection of both vertical and horizontal contamination depending on the placement of the tracers relative to the test samples. The devices of the present invention that are manufactured to contain tracers allow the end user, once the assay is complete, to evaluate each well to determine which tracers are present. Detection of a tracer in a particular well that was not originally in the well indicates cross contamination.

In one such embodiment of a device of the invention, the device is a multiwell plate comprising wells that contain a plurality of calibration standard and a plurality of internal standard (CS+IS), wells that contain a plurality of internal standard only (IS), wells that contain a quality control standard and an internal standard (QC+IS), wells that contain one or a plurality of tracers, and optionally, wells that are empty (blank).

The ability to easily configure the devices of the present disclosure allows for customization according to the particular end user's requirements. The number of tests that may be performed with each device will vary depending upon the number of wells or vials that contain quality control standards, calibration standards or are left blank. The standards and the number of wells or vials comprising the standards may vary depending on the particular assays to be performed.

Use of the Devices of the Present Disclosure

The devices of the presently disclosed invention simplify the task of preparing test samples for quantitative analysis for the end user of the devices. The device is precisely manufactured to yield consistent results and to reduce the error that can accompany sample preparation.

The end user of a device of the present invention will provide a test sample or an analyte to be tested or quantified. The test sample can be a biological sample such as urine, serum plasma, blood, saliva, cerebral spinal fluid, feces, semen, or vaginal fluid. After preparation of the test sample using an isolation, concentration or purification step as described herein, the test sample can be tested directly in some applications but may also be further purified or extracted prior to analysis by any suitable method. Such methods are well known in the art, for example, liquid phase extraction, solid phase extraction and high-performance liquid chromatography (HPLC). Analysis can be performed by any suitable method, such methods are well known in the art, for example gas chromatography (GC), quantitative mass spectrometry tandem mass spectroscopy (MS/MS), liquid chromatography-electrospray tandem mass spectrometry (LC-MS/MS), or liquid chromatography-electrospray time-of-flight mass spectrometry. In other embodiments, analysis of the extracted test sample can be performed by any quantitative analytical method, for example, a mass spectrometric method, an electrophoretic method, NMR, a chromatographic method or a combination thereof. In a further embodiment, the mass spectrometric method is LC-MS and LC-MS/MS. In some embodiments, the LC-MS/MS can be performed using LC-Orbitrap, LC-FTMS, LC-LTQ, MALDI-MS including but not limited to MALDI-TOF, MALDI-TOF/TOF, MALDI-qTOF, and MALDI-QIT. Preferably, the mass spectrometric method is a quantitative MALDI-MS or LC-MS using optimized conditions. In still another embodiment, the electrophoretic method is CE-LIF. In yet another embodiment, methods such as capillary gel electrophoresis or capillary zone electrophoresis can be used with the inventive methods.

The methods of the present invention do not include steps of preparing or adding the calibration, quality control, or internal standards, to the wells or vials of the device by the end user. The presently disclosed devices are manufactured to contain precisely controlled amounts of calibration standards, quality control standards and internal standards in dried form as appropriate for the quantitative analysis of a plurality of analytes of interest in a test sample.

One embodiment of a method “A” of the present disclosure is a method for determining the concentration of one or more analytes in one or a plurality of test samples comprising the steps: i) providing a device of the present invention wherein the device is a multiwell plate comprising a plurality of wells containing a plurality of calibration standards and a plurality of internal standards (CS+IS), a plurality of wells containing a plurality of quality control standards and a plurality of internal standards (QC+IS), and a plurality of wells containing a plurality of internal standards (IS); ii) adding a blank sample into each of the wells containing the (CS+IS) and into each of the wells containing the (QC+IS); adding a test sample into a well containing (IS); iv) mixing the well contents to generate a plurality of well samples; v) preparing the well samples for quantitative analysis; vi) quantitative analysis of the well samples; and vii) determining the concentration of the analytes present in the test samples, wherein steps ii) and iii) can be performed in any order.

In one embodiment of method A, the blank sample of step comprises a solvent system, a biological matrix, or a combination thereof. In some embodiments, the solvent system of step ii) comprises water, an aqueous solvent system, an organic solvent, or any combination thereof. In one embodiment of method A, the blank sample of step comprises an aqueous solvent system wherein the aqueous solvent system is an aqueous buffer. In an embodiments of method A, the blank sample comprises a solvent system wherein the solvent system comprises an organic solvent selected from the group consisting of methanol, ethanol, isopropanol, acetonitrile, dimethylsulfoxide, N,N-dimethylformamide, and any combination thereof. In another embodiment the solvent system is water. In some embodiments of method A, the blank sample comprises a biological matrix selected from the group consisting of urine, serum, plasma, blood, saliva, cerebral spinal fluid, feces, semen, or vaginal fluid. In another embodiment of method A, the blank sample comprises a biological matrix wherein the biological matrix is urine. In yet another embodiment of method A, the blank sample comprises a biological matrix wherein the biological matrix is blood. In still another embodiment of method A, the blank sample comprises a biological matrix wherein the biological matrix is plasma.

In an embodiment of method A, the test sample is a substance obtained from a test subject wherein the test subject is a mammal. In another embodiment the test sample is obtained from a horse, cow, pig, cat, dog, non-human primate or a human. In yet another embodiment the test subject is a human. In some embodiments the substance taken from the test subject selected from the group consisting of urine, serum, plasma, blood, saliva, cerebral spinal fluid, feces, semen, or vaginal fluid. In one embodiment of method A, the test sample is urine. In yet another embodiment of method A, the test sample is blood. In still another embodiment of method A, the test sample is plasma.

In some embodiments, step iii) further comprises adding a solvent system to the test sample. In some embodiments, the solvent system added to the test samples selected from the group consisting of water, an aqueous solvent system, an organic solvent, or any combination thereof. For example, in one embodiment of method A, step iii) further comprises the addition of an aqueous solvent system wherein the aqueous solvent system comprises an aqueous buffer. In another embodiment the solvent system comprises an organic solvent selected from the group consisting of methanol, ethanol, isopropanol, acetonitrile, dimethylsulfoxide, N,N-dimethylformamide, and any combination thereof.

In some embodiments of method A, the preparing the well samples of step v) comprises parallel concentration means to prepare the well samples for quantitative analysis. In some embodiments, the parallel concentration means to prepare the well samples of step v) can include, techniques to isolate, concentrate or purify the target analyte in the well samples prior to the quantitative analysis step. Exemplary parallel concentration means can include solid phase extraction, liquid phase extraction, chromatography, and any combination thereof. In one embodiment the parallel concentration means comprises solid phase extraction.

In some embodiments, the quantitative analysis of step vi) comprises separation of analytes in each well after the preparation of the well samples in step v). In some embodiments, the separation of the analytes comprises liquid chromatography. In one embodiment, the separation of analytes comprises high-performance liquid chromatography (HPLC). In some embodiments, the quantitative analysis of step vi) comprises analysis of separated analytes by any quantitative analytical method, for example, a mass spectrometric method, an electrophoretic method, NMR, a chromatographic method or a combination thereof. In some embodiments, the analysis of the analytes can be preformed using quantitative mass spectrometry. In some embodiments, the quantitative mass spectrometry of step vi) comprises positive electrospray ionization mass spectrometry. In one embodiment, the quantitative analysis of step vi) comprises liquid chromatography-electrospray tandem mass spectrometry (LC-MS/MS). In one embodiment, the quantitative analysis of step vi) comprises liquid chromatography-positive electrospray ionization LC-MS/MS. In one embodiment, the quantitative analysis of step vi) comprises liquid chromatography-negative electrospray ionization LC-MS/MS.

Kits

The presently disclosed kits may be used to quantify analytes in a test sample. One embodiment of a kit of the present invention includes a kit comprising: a device of the present invention and a detailed written description of the specifications of the device and instructions for using the device to perform the chemical analysis and quantification of one or more analytes. In yet another embodiment the kit comprises a device wherein the device is a plurality of vials according to the invention and a detailed written description of the specifications of the device. In another embodiment the kit comprises a device wherein the device is a multiwell plate according to the invention and a detailed written description of the specifications of the device wherein the detailed written description provides the precise amounts of the components in each well or vial of the device. In still another embodiment the kit comprises a device wherein the device is plurality of vials according to the invention and a detailed written description of the specifications of the device wherein the detailed written description provides the precise amounts of the components in each well or vial of the device.

In some embodiments, the kits of the present invention comprises a plurality of devices as described herein. In some embodiments, the kit includes a device, for example, a 96 well multiwell plate. In another embodiment of the kit, the device is a 384 well multiwell plate. In another embodiment of the kit, the device is a 1024 well multiwell plate. In another embodiment of the kit, the device is a 1536 well multiwell plate. In another embodiment of the kit, the device is a 50, 100, 150, 200, or 300 vial tray or array.

In some embodiments the kit further comprises a solid phase extraction device. In one embodiment the kit comprises a solid phase extraction device where in the solid phase extraction device is a supported liquid extraction plate or column. In another embodiment the kit comprises a supported liquid extraction device wherein the supported liquid extraction device is an Isolute® SLE⁺96 well plate or Isolute® SLE⁺ column, Biotage, Charlotte, N.C.

In some embodiments the kit further compromises a deconjugation plate. The deconjugation plate is a 96 or 384 well plate that contains the beta-glucuronidase enzyme or other deconjugation enzymes as a dry residue in the sample wells.

In any of the kits of the invention, the kit may further comprise standard operating procedures for measuring specific analytes in human urine or blood wherein the procedures are customized to meet specific end user validation requirements.

### EXAMPLES

The following examples are for illustrative purposes only and are not intended to limit the scope of the present invention. The devices of the present invention are highly customizable so as to meet the requirement of a variety of end users.

### Example 1: Customized Device for the Quantification of Multiple Drugs

TABLE 1

| Ref. | 1 | 2 | 3 | 4 | 5 | 6 | 7 | 8 | 9 | 10 | 11 | 12 |
|---|---|---|---|---|---|---|---|---|---|---|---|---|
| A | CS + IS | QC + IS | IS | IS | IS | IS | IS | IS | IS | IS | QC + IS | Blank |
| B | CS + IS | QC + IS | IS | IS | IS | IS | IS | IS | IS | IS | QC + IS | Blank |
| C | CS + IS | QC + IS | IS | IS | IS | IS | IS | IS | IS | IS | QC + IS | Blank |
| D | CS + IS | QC + IS | IS | IS | IS | IS | IS | IS | IS | IS | QC + IS | Blank |
| E | CS + IS | QC + IS | IS | IS | IS | IS | IS | IS | IS | IS | QC + IS | Blank |
| F | CS + IS | QC + IS | IS | IS | IS | IS | IS | IS | IS | IS | QC + IS | Blank |
| G | CS + IS | QC + IS | IS | IS | IS | IS | IS | IS | IS | IS | QC + IS | Blank |
| H | CS + IS | QC + IS | IS | IS | IS | IS | IS | IS | IS | IS | QC + IS | Blank |

CS: calibration standard
QC: quality control standard
IS: internal standard

Table 1 represents an exemplary configuration of wells in a particular embodiment of a device of the present invention manufactured for the quantification multiple drugs in a plurality of test samples. Each well is referred to using the row reference with the column reference. For example the well in uppermost row and in the left most column is referred to as A1. The plates are manufactured by adding, to the wells of the plate, specific amounts of a calibration standard mix, a quality control standard mix, an internal standard spiking mix or a combination thereof. The procedure for making the device of Example 1 is described below.

The calibration standard mix comprises calibration standards for each analyte to be quantified. The calibration standard mix is added in an amount such that the wells comprise amounts of the calibration standards at 0.25, 0.5, 1.25, 2.5, 5.0, 12.5, 25 and 50 ng. Specifically wells A1 and B1 are prepared by adding 50 or 100 μl, of a 0.005 μg/mL calibration standard mix to the respective locations; wells C1, D1, and E1 are prepared by adding 25, 50, and 100 μL of a 0.05 μg/mL calibration standard mix to the respective locations; and wells F1, F2, and F3 are prepared by adding 25, 50, and 100 μL of a 0.5 μg/mL calibration standard mix to the respective locations.

The quality control standard mix comprises quality control standards for each analyte to be quantified. The quality control mix is added to the wells of the plate such that the wells comprise amounts of the quality control standards at 0.25, 0.5, 1.25, 2.5, 5.0, 12.5, 25 and 50 ng. Specifically, wells A2 and B2 are prepared by adding 50 or 100 μL of a 0.005 μg/mL standard mix to the respective locations; wells C2, D2, and E2, are prepared by adding 25, 50, and 100 μL of a 0.05 μg/mL quality control standard mix to the respective locations; and wells F2, G2, and H2 are prepared by adding 25, 50, and 100 μL of a 0.5 μg/mL quality control standard mix to the respective locations. The wells of A11-H11 are loaded in the same manner as corresponding wells A2-H2.

25 μL of internal standard spiking mix is added to all wells of the plate with the exception of row 12 (blanks). The internal standard spiking mix comprises internal standards at varying concentrations 12.5 to 37.5 ng depending on the particular internal standard.

Suitable reference standard mixes can be obtained from commercial sources for example from Fisher Scientific, Pittsburgh, Pa.; Sigma-Aldrich, St. Louis, Mo.; Cayman Chemical, Ann Arbor, Mich.; Cerriliant, Round Rock, Tex.; Cambridge Isotopes, Tewksbury, Mass. or Lipomed, Cambridge, Mass.

### Example 2: Customized Device for the Quantification of Multiple Drugs

TABLE 2

| Ref. | 1 | 2 | 3 | 4 | 5 | 6 | 7 | 8 | 9 | 10 | 11 | 12 |
|---|---|---|---|---|---|---|---|---|---|---|---|---|
| A | CS + IS | CS + IS | CS + IS | CS + IS | CS + IS | CS + IS | CS + IS | CS + IS | CS + IS | CS + IS | CS + IS | Blank |
| B | QC + IS | IS | IS | IS | IS | IS | IS | IS | IS | IS | IS | Blank |
| C | QC + IS | IS | IS | IS | IS | IS | IS | IS | IS | IS | IS | Blank |
| D | QC + IS | IS | IS | IS | IS | IS | IS | IS | IS | IS | IS | Blank |
| E | QC + IS | IS | IS | IS | IS | IS | IS | IS | IS | IS | IS | Blank |
| F | QC + IS | IS | IS | IS | IS | IS | IS | IS | IS | IS | IS | Blank |
| G | QC + IS | IS | IS | IS | IS | IS | IS | IS | IS | IS | IS | Blank |
| H | QC + IS | IS | IS | IS | IS | IS | IS | IS | IS | IS | IS | Blank |

The table above represents the configuration of wells in a particular embodiment of a device of the present disclosure prepared for the quantification multiple drugs in a plurality of test samples. The represented device is prepared essentially as described in Example 1 except the device is customized to allow for more wells that contain CS+IS, less wells that contain QC+IS and more wells that contain IS only.

### Example 3: Customized Device for Quantifying Multiple Drugs from Urine Samples

The device may be configured in any suitable manner, for the quantification of multiple drugs, for example as described in Examples 1 and 2. In addition to the calibration standard mix, quality control standard mix, and internal standard spiking mix, 1254 of 0.1M pH 4 ammonium acetate buffer and 50 μl β-glucuronidase (100,000 units/mL) are added to each well of the device.

### Example 4: Customized Device to Detect Cross-Well Contamination

TABLE 3

| Ref. | 1 | 2 | 3 | 4 | 5 | 6 | 7 | 8 | 9 | 10 | 11 | 12 |
|---|---|---|---|---|---|---|---|---|---|---|---|---|
| A | (1, 3) | (2, 3) | (1, 3) | (2, 3) | (1, 3) | (2, 3) | (1, 3) | (2, 3) | (1, 3) | (2, 3) | (1, 3) | (2, 3) |
| B | (1, 4) | (2, 4) | (1, 4) | (2, 4) | (1, 4) | (2, 4) | (1, 4) | (2, 4) | (1, 4) | (2, 4) | (1, 4) | (2, 4) |
| C | (1, 3) | (2, 3) | (1, 3) | (2, 3) | (1, 3) | (2, 3) | (1, 3) | (2, 3) | (1, 3) | (2, 3) | (1, 3) | (2, 3) |
| D | (1, 4) | (2, 4) | (1, 4) | (2, 4) | (1, 4) | (2, 4) | (1, 4) | (2, 4) | (1, 4) | (2, 4) | (1, 4) | (2, 4) |
| E | (1, 3) | (2, 3) | (1, 3) | (2, 3) | (1, 3) | (2, 3) | (1, 3) | (2, 3) | (1, 3) | (2, 3) | (1, 3) | (2, 3) |
| F | (1, 4) | (2, 4) | (1, 4) | (2, 4) | (1, 4) | (2, 4) | (1, 4) | (2, 4) | (1, 4) | (2, 4) | (1, 4) | (2, 4) |
| G | (1, 3) | (2, 3) | (1, 3) | (2, 3) | (1, 3) | (2, 3) | (1, 3) | (2, 3) | (1, 3) | (2, 3) | (1, 3) | (2, 3) |
| H | (1, 4) | (2, 4) | (1, 4) | (2, 4) | (1, 4) | (2, 4) | (1, 4) | (2, 4) | (1, 4) | (2, 4) | (1, 4) | (2, 4) |

Table 3 shows one aspect of embodiment of a device of the present disclosure that allows for monitoring and detection of cross-well contamination. The device may be configured in any suitable manner, for the quantification of multiple drugs, for example as described in Examples 1, 2 and 3. In addition to the calibration standard mix, quality control standard mix, and internal standard spiking mix, four tracers are added in a configuration that allows detection of carryover of a tracer from one well to another well. Table 3 shows one possible configuration of the tracers for a 96-well plate. The numbers 1-4 are used to represent the four tracers, hydrocodone-d3 (1), methadone-d3 (2), amphetamine-d5 (3) and codeine-d3 (4) respectively. In this configuration, each well contains two tracers, for example (1,3) indicates that the well contains tracer 1 and 3 and (2,4) indicates that the well contains tracer 2 and 4.

### Example 5: Customized Device to Detect Cross-Well Contamination

A multiwell plate is configured as shown in Table 4 and the four tracers are Amphetamine-d5 (1), Amphetamine-d6 (2), Amphetamine-d8 (3) and Amphetamine-d10 (4). An alternate configuration is also possible where non-labeled analytes are used if not being used for quantitation. For example acetyl fentanyl (1), acetyl fentanyl-d5 (2), acetyl norfentanyl (3), and acetyl norfentanyl-d5 (4) may be used.

As proof of concept example acetyl fentanyl (1), acetyl fentanyl-d5 (2), acetyl norfentanyl (3), and acetyl norfentanyl-d5 (4) were manufactured in 96 well plates in the orientation noted in Table 3. In brief, 500 ng of each tracer were contained in each well as a dry residue. Urine samples used for normal drug analysis were incorporated following standard drug analysis protocols. Urine samples were then purposely contaminated with some of the contents from adjacent wells in both horizontal and vertical fashion to measure the ability of each tracer to guard against well-to-well contamination. After purposely contaminating urine samples, samples contained within each well were evaluated using LC-ToF-MS techniques for the presence of tracers originating from adjacent wells not purposely incorporated in the well being evaluated. In addition, contamination was evaluated at different steps used in sample preparation protocols. Specifically, sample were contaminated prior to incubation, prior to extraction, after extraction and after reconstitution steps. In each case tracers adequately controlled for cross contamination and was able to detect cross contamination when wells were purposefully contaminated with 1-10% volume of adjacent wells.

### Example 6

Devices of the present disclosure can be manufactured according to the end user's specific needs. This example describes a customized device which is manufactured for the quantitative determination of the concentration of 52 drugs in one or a plurality of test samples. The plates are configured according to Example 1. The wells designated CS+IS and the wells designated QC+IS each contain precise pre-determined amounts of each of the analytes listed in Table 4. In general, the amount of the analyte used in the varying amounts of the calibration standards required to generate the calibration curve for that analyte will be selected so that the calibration curve includes the therapeutic range of each of the analytes tested. The quality control standards will generally cover the same range of concentration of the analyte as the calibration standards.

TABLE 4
Analytes

| | | Therapeutic |
|---|---|---|
| | | Range* |
| Analyte | Trade or Other Names | (ng/mL) |
| 2-ethylidene-1,5-dimethyl-3,3- | EDDP, metabolite of | N/A |
| diphenylpyrrolidine | methadone | |
| 3,4-methylenedioxyamphetamine | MDA | <400 |
| 3,4-methylenedioxyethylamphetamine | MDEA | <200 |
| 3,4-methylenedioxymethylamphetamine | MDMA, Ecstasy | 100-350 |
| 6-monoacetylmorphine | 6-MAM, 6-acetylmorphine | N/A |
| 7-aminoclonazepam | metabolite of clonazepam | N/A |
| Acetyl fentanyl | n/a | N/A |
| Acetyl norfentanyl | metabolite of acetyl | N/A |
| | fentanyl | |
| Alprazo lam | Xanax, Niravam | 5-50 |
| Amphetamine | Adderall | 20-100 |
| Benzoylecgonine | metabolite of cocaine | <100 |
| Buprenorphine | Buprenex, Butrans | 0.5-5 |
| Carisoprodol | Soma | 10000-30000 |
| Clonazepam | Klonopin | 20-80 |
| Cocaine | n/a | 50-300 |
| Codeine | 3-methylmorphine, | 30-250 |
| | contained in Cheracol, | |
| | Robitussin A-C, | |
| Cyclobenzaprine | Flexeril, Amrix, Fexmid | 3-40 |
| Dextromethorphan | Delsym | 10-40 |
| Diazepam | Valium, Diastat | 100-2000 |
| Dihydrocodeine | Drocode, Paracodeine, | 30-250 |
| | Parzone, Paramol | |
| Fentanyl | Duragesic, Actiq, Abstral, | 3-300 |
| | Lazanda, Subsys, Fentora, | |
| | Sublimaze, Onsolis, Ionsys | |
| Flunitrazepam | Rohypnol | 5-15 |
| Flurazepam | Dalmane, Dalmadorm | 20-100 |
| Heroin | 3,6-diacetylmorphine | N/A |
| Hydrocodone | contained in Lorcet, | 10-50 |
| | Loritab, Norco, Vicodin, | |
| | Hycodan, Tussionex | |
| Hydromorphone | Dilaudid, Exalgo, | 5-50 |
| | Palladone | |
| Lorazepam | Ativan | N/A |
| Meperidine | Demerol | 100-800 |
| Meprobamate | Miltown, Equanil, | 5000-10000 |
| | Meprospan | |
| Methadone | Diskets, Methadose | 10-1000 |
| Methamphetamine | Desoxyn | <100 |
| Midazolam | Versed, Hypnovel, | 40-100 |
| | Dormicum | |
| Morphine | Oramorph, MS Contin, | 10-300 |
| | Avinza, Kadian, Roxanol, | |
| | Kapanol | |
| Naloxone | Narcan | 10-30 |
| Naltrexone | Revia, Vivitrol | 3-50 |
| Nitrazepam | Alodorm, Arem, Insoma, | N/A |
| | Mogadon, Nitrados, | |
| | Somnite | |
| Norbuprenorphine | metabolite of | N/A |
| | buprenorphine | |
| Nordiazepam | metabolite of diazepam | 200-800 |
| Norfentanyl | metabolite of fentanyl | N/A |
| Normeperidine | metabolite of meperidine | N/A |
| Norpropoxyphene | metabolite of | N/A |
| | propoxyphene | |
| Oxazepam | Alepam, Medopam, | N/A |
| | Murelax, Noripam, | |
| | Opamox, Serepax | |
| Oxycodone | Roxycodone, Oxycontin, | 5-100 |
| | Oxceta | |
| Oxymorphone | Opana, Numorphan | N/A |
| Phenazepam | n/a | N/A |
| Phencyclidine | PCP | 10-200 |
| Phentermine | Adipex-P, Suprenza, | 30-100 |
| | Ionamin | |
| Propoxyphene | Darvon, Dolene | 50-300 |
| Sufentenil | Sufenta | 0.5-10 |
| Temazepam | Restoril, Normison | 0.2-150 |
| Tramadol | Ultram, ConZip, Ryzolt | 100-1000 |
| α-Hydroxyalprazolam | metabolite of alprazolam | N/A |

*The therapeutic ranges for the drugs are from in Schulz, et al. 2012, Critical Care vol. 16 (R136) pages 1-4.

The wells designated with IS, CS+IS, or QC+IS each contain a precise amount of: 7-aminoclonazepam-d4, Alprazolam-d5, Clonazepam-d4, Diazepam-d5, Flunitrazepam-d3, α-hydroxyalprazolam-d5, Lorazepam-d4, Midazolam-d4, Nitrazepam-d5, Nordiazepam-d5, Oxazepam-d5, Phenazepam-d4, Temazepam-d5, Amphetamine-d11, Benzoylecgonine-d3, Carisoprodol-d7 Cocaine-d3, Cyclobenzaprine-d3, MDA-d5, MDEA-d5, MDMA-d5, Methamphetamine-d5, Phencyclidine-d5, Phencyclidine-d5, Phentermine-d5, 6-MAM-d6, Acetyl fentanyl-d5, Acetyl norfentanyl-d5, Buprenorphine-d4, Codeine-d6, Dextromethorphan-d3, Dihydrocodeine-d6, EDDP-d3, Fentanyl-d5, Heroin-d9, Hydrocodone-d6, Hydromorphone-d3, Meperidine-d4, Meprobamate-d7, Methadone-d9, Morphine-d3, Naloxone-d5, Naltrexone-d3, Norbuprenorphine-d3, Norfentanyl-d5, Normeperidine-d4, Norpropoxyphene-d5, Oxycodone-d6, Propoxyphene-d5, Sufentenil-d5, and Tramadol-13C-d3.

### Example 7: Quantitative Analysis of Multiple Drugs in Blood by LC-MS/MS

The method described in this example is for exemplary purposes. Other methods of quantification are well known to one skilled in the art and are suitable for use in conjunction with the devices of the present disclosure.

Test samples (blood) are obtained from test subjects. To a plate configured according to Example 1, a blank sample is added to the wells identified as CS+IS, QC+IS or blank. The blank sample comprises 500 μl of a blood sample that does not contain any of the analytes to be quantified, 500 μl of 0.5 molar ammonium hydroxide buffer, and 125 μl of acetonitrile. 500 μl of test samples in duplicate are added to wells identified as IS along with 500 μl of 0.5 molar ammonium hydroxide buffer and 125 μl of acetonitrile; the contents of the wells are mixed for 10 min in an orbital shaker. After mixing, the contents of the wells are processed in parallel by loading the contents into corresponding wells of a supported liquid extraction plate (e.g., a Isolute® SLE plate, Biotage Charlotte, N.C.). The analytes are eluted, dried and reconstituted in methanol (100 μl) The processed samples are then analyzed by positive electrospray ionization LC-MS/MS.

### Example 8: Quantitative Analysis of Multiple Drugs in Urine by LC-MS/MS

Test samples (urine) are obtained from test subjects. To a plate configured according to Example 1, a blank sample is added to the wells identified as CS+IS, QC+IS or blank. The blank sample comprises 500 μl of a urine sample that does not contain any of the analytes to be quantified, 487 μl of pH5 0.1 molar ammonium acetate buffer, and 13 μl of β-glucuronidase enzyme (5000 units/mL). 500 μl of test samples in duplicate are added to wells identified as IS along with 487 μl of pH5, 0.1 molar ammonium acetate buffer, and 13 μl of β-glucuronidase enzyme (5000 units/mL); the contents of the wells are simultaneously incubated at 37° C. mixed for 30 min in an orbital shaker. After mixing, 500 μl 0.5 molar ammonium hydroxide buffer is added to all wells, and the contents of the wells are processed in parallel by loading the contents into corresponding wells of a supported liquid extraction plate (e.g., a Isolute® SLE plate, Biotage Charlotte, N.C.). The analytes are eluted, dried and reconstituted in methanol (100 μl) The processed samples are then analyzed by positive electrospray ionization LC-MS/MS

### Example 9: Precision

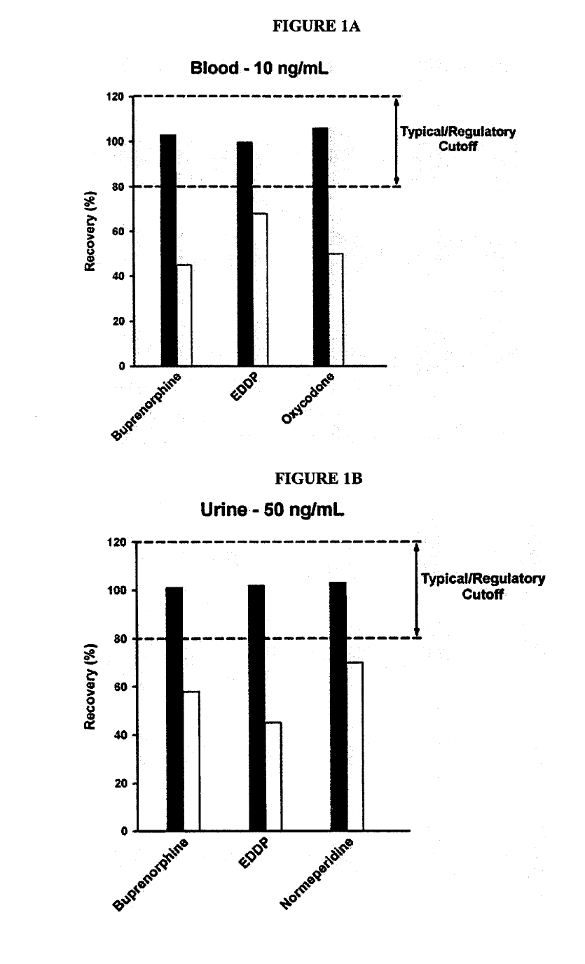

Rigorous quality control studies have been performed using devices of the invention that are multiwell plates where calibration standards and quality control standards were prepared at multiple concentration for each of 52 analytes (listed in Table 3) the well samples were prepared in the same manner as for a test sample and concentrations of the analytes were determined by LC-MS/MS. The percentage of drug recovered from the well samples consistently met typical regulatory requirements. FIGS. 1A and 1B show the percent recovery for three of the drugs, buprenorphine, EDDP and oxycodone where the blank matrix was blood (FIG. 1A) or urine (FIG. 1B). The data for buprenorphine, EDDP and oxycodone are representative of the 52 analyte tested. The black bars represent the recovery using a device of the present invention whereas the white bars represent data published by Biotage in an application note AN790 that is available at the website: http://data.biotage.co.jp/pdf/application/AN790.pdf. FIGS. 1A and 1B of the present disclosure shows that in urine or blood the device of the invention gave recoveries well within the typical FDA regulatory requirements as set out by the dotted lines. In contrast, the data published by Biotage the recoveries were significantly lower that what is generally required by the FDA.

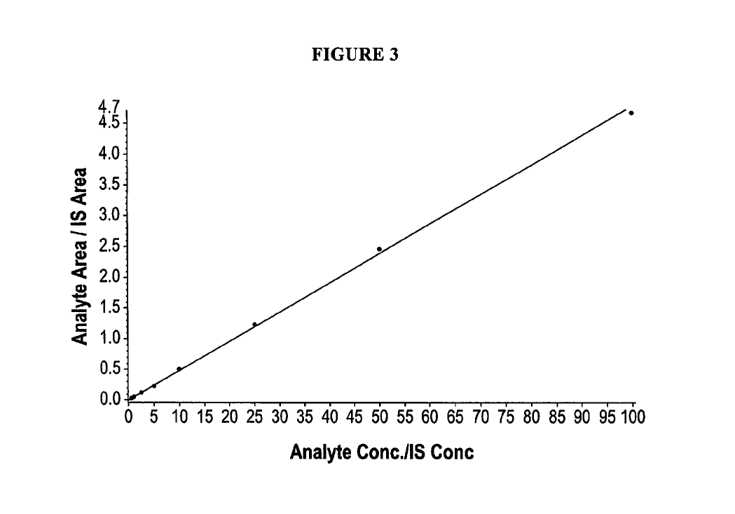

The precision of the device is further illustrated in a standard curve of acetyl fentanyl prepared using the device of the present invention. The data in FIG. 3 was acquired as part of the QC process for a custom panel of opiates, benzodiazepines, and several drugs of abuse. The calibration, quality control, and internal standards wells were reconstituted in 500 μL of MeOH and gently vortexed for 1 min. The plate was placed in the autosampler, held at a temperature of 4° C., and interfaced with an Agilent 1100 series HPLC. The samples were eluted with mobile phase A: 10 mM ammonium formate in MilliQ water, and mobile phase B: 0.1% formic acid in MeOH. A Phenomenex, Kinetex hexylphenyl, 4.6 mm×100 mm, 2.6 micron particle size, 100 angstrom column was used. The HPLC was coupled to an ABSciex 4000 triple quad MS with a TurboV ion spray (+) ESI ion source. The results of FIG. 3 detail the linearity of one of 51 analytes: acetyl fentanyl. The linearity of the calibration standards, the accuracy of the second source QC standards, and the precision of the internal standards are shown. The graphical results of the standard curve using acetyl fentanyl are depicted in FIG. 3.

TABLE 5
Standard curve of acetyl fentanyl (0-100 ng/mL) using the
device of the present invention

| | Assigned | Concentration | Recovery |
|---|---|---|---|
| | Value (ng) | (ng/mL) | (Pass +/−25%) |
| Blank IS | N/A | 7.58E+05 | |
| Blank IS | N/A | 7.06E+05 | |
| Blank IS | N/A | 7.17E+05 | |
| Blank IS | N/A | 7.44E+05 | |
| QC1 | 2.5 | 2.56 | 102.4% |
| QC1 | 2.5 | 2.45 | 98.0% |
| QC1 | 2.5 | 2.56 | 102.4% |
| QC1 | 2.5 | 2.72 | 108.8% |
| QCL | 2.5 | 2.64 | 105.6% |
| QC2 | 10 | 10.5 | 105.0% |
| QC2 | 10 | 10.5 | 105.0% |
| QC2 | 10 | 10.2 | 102.0% |
| QC2 | 10 | 10.6 | 106.0% |
| QC2 | 10 | 10.6 | 106.0% |
| QC3 | 25 | 24 | 96.0% |
| QC3 | 25 | 25.2 | 100.8% |
| QC3 | 25 | 24.4 | 97.6% |
| QC3 | 25 | 25.6 | 102.4% |
| QC3 | 25 | 25.7 | 102.8% |
| QC4 | 100 | 101 | 101.0% |
| QC4 | 100 | 94.4 | 94.4% |
| QC4 | 100 | 105 | 105.0% |
| QC4 | 100 | 98.4 | 98.4% |
| QC4 | 100 | 103 | 103.0% |

The calculated correlation coefficient was determined to be: y = 0.0476 + 0.000986 (r = 0.9996)

### Example 10: Deconjugation Plates

The method described in this example is for exemplary purposes. Other methods of quantification are well known to one skilled in the art and are suitable for use in conjunction with the devices of the present disclosure. Urine samples samples obtained from test subjects and are added to a plate coated with the beta-glucuronidase enzyme, with or without a buffer solution. The deconjugation plate is created by the addition of the appropriate amount of enzyme to each well, typically 5-10K units, either as a solution in water or buffer. The aqueous enzyme solutions may be transformed to neat residue by evaporation or lyophilization.

Experimental Procedure

The deconjugation plate was constructed and tested using glucuronide conjugates of morphine. 96 well plates were manufactured with β-glucuronidase derived from limpets (Patella vulagata). Each well was specifically tittered to have approximately 13,000 activity units and lyophilized to dryness. For comparison, some wells were processed following standard procedures that externally reconstitute β-glucuronidase derived from limpets (Patella vulagata). The standard procedure dilutes the enzyme in a manner to deliver approximately 13,000 activity units in 153 μL. Therefore, 153 μL of this enzyme mixture was added to blank wells to directly compare a traditional standard procedure used herein with premanufactured deconjuation plates constructed herein. All wells from both the premanufactured deconjugation plate and the standard procedure plate were then diluted with 0.1M pH5 ammonium acetate buffer (0.5 mL final volume). Test urine specimens were created by spiking morphine-3β-D-glucuronide (500 ng/mL) in test samples known to be free of this morphine metabolite. The test urine sample was incorporated into 96 wells containing β-glucuronidase pH 5, and incubated following standard procedures at 60° C. for various time points. The formation of morphine using standard LC-ToF-MS analytical techniques was used to measure enzyme activity. Under these conditions and with normal procedures maximum cleavage occurs at 180 min.

Results

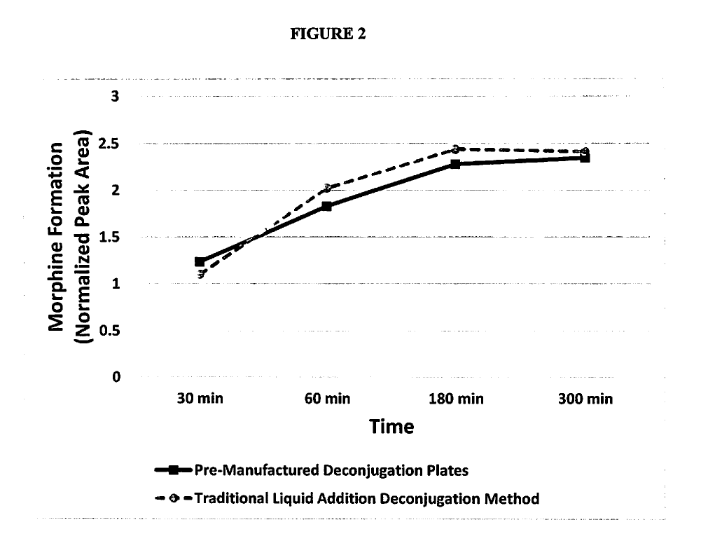

As shown in FIG. 2, no significant difference in deconjuation rates were observed in samples processed in wells premanufactured with β-glucuronidase derived from limpets (Patella vulagata) when compared to samples processed following standard procedures.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A device for quantifying the concentration of a plurality of analytes in a test sample, the device comprising:
a multi-well plate wherein each well independently is left empty or independently comprises a dried calibration standard, a dried quality control standard, a dried internal standard, or any combination thereof; or
a plurality of vials wherein each vial is left empty or independently comprises a calibration standard, a quality control standard, an internal standard, or any combination thereof.

2. The device according to claim 1, wherein the calibration standard is from a different source than the quality control standard.

3. The device according to claim 1, wherein at least one calibration standard is an analyte selected from the group consisting of norpropoxyphene, propoxyphene, amphetamine, 3,4-methylenedioxyamphetamine (MDA), 3,4-methylenedioxy-N-ethylamphetamine (3,4-MDEA), 3,4-methylenedioxy-N-methylamphetamine (3,4-MDMA), methadone, methamphetamine, 6-monoacetylmorphine (6-MAM), 7-aminoclonazepam, α-hydroxyalprazolam, acetyl fentanyl, acetyl norfentanyl, alprazolam, benzoylecgonine, buprenorphine, carisoprodol, clonazepam, cocaine, codeine, cyclobenzaprine, dextromethorphan, diazepam, dihydrocodeine, 2-ethylidene-1,5-dimethyl-3,3-diphenylpyrrolidine (EDDP), fentanyl, flunitrazepam, flurazepam, heroin, hydrocodone, hydrodone, hydromorphone, lorazepam, meperidine, meprobamate, midazolam, morphine, naloxone, naltrexone, nitrazepam, norbuprenorphine, nordiazepam, norfentanyl, normeperidine, oxazepam, oxycodone, oxymorphone, phenazepam, phencyclidine (PCP), phentermine, sufentanil, temazepam, cis-tramadol, ecgonine methyl ester, anhydroecgonine methyl ester, cocaethylene, 6-acetylcodeine, N-methyl-1,3-benzodioxolylbutanamine (MBDB), mephedrone, normephodrone, cathinone, Δ9-tetrahydrocannabinol (Δ9-THC), tetrahydrocannabinolic acid, ketamine, gabapentin, pregabalin, and norsufentanil.

4. The device according to claim 1, wherein at least one of the quality control standards is selected from the group consisting of norpropoxyphene, propoxyphene, amphetamine, 3,4-methylenedioxyamphetamine (MDA), 3,4-methylenedioxy-N-ethylamphetamine (3,4-MDEA), 3,4-methylenedioxy-N-methylamphetamine (3,4-MDMA), methadone, methamphetamine, 6-monoacetylmorphine (6-MAM), 7-aminoclonazepam, α-hydroxyalprazolam, acetyl fentanyl, acetyl norfentanyl, alprazolam, benzoylecgonine, buprenorphine, carisoprodol, clonazepam, cocaine, codeine, cyclobenzaprine, dextromethorphan, diazepam, dihydrocodeine, 2-ethylidene-1,5-dimethyl-3,3-diphenylpyrrolidine (EDDP), fentanyl, flunitrazepam, flurazepam, heroin, hydrocodone, hydrodone, hydromorphone, lorazepam, meperidine, meprobamate, midazolam, morphine, naloxone, naltrexone, nitrazepam, norbuprenorphine, nordiazepam, norfentanyl, normeperidine, oxazepam, oxycodone, oxymorphone, phenazepam, phencyclidine (PCP), phentermine, sufentanil, temazepam, cis-tramadol, ecgonine methyl ester, anhydroecgonine methyl ester, cocaethylene, 6-acetylcodeine, N-methyl-1,3-benzodioxolylbutanamine (MBDB), mephedrone, normephodrone, cathinone, Δ9-tetrahydrocannabinol (Δ9-THC), tetrahydrocannabinolic acid, ketamine, gabapentin, pregabalin, and norsufentanil.

5. The device according to claim 1, wherein the device is a multi-well plate wherein the multi-well plate is a 48 well plate, a 96 well plate, a 384 well plate or a 1536 well plate.

6. The device according to claim 1, wherein the plurality of internal standards are each independently a reference standard of an analyte to be quantified in the test sample, wherein the reference standard has an atom that is substituted.

7. The device according to claim 6, wherein the atom is substituted with an isotope of the substituted atom.

8. The device according to claim 7, wherein the substituted atom is hydrogen and the isotope is deuterium.

9. The device according to claim 7, wherein the substituted atom is carbon.

10. The device according to claim 9, wherein the isotope is carbon-12, carbon-13, carbon-16 or carbon-18.

11. The device according to claim 6, wherein the substituted atom is nitrogen.

12. The device according to claim 11, wherein the isotope is nitrogen-14 or nitrogen-15.

13. The device according to claim 6, wherein the substituted atom is hydrogen and the hydrogen is substituted with fluorine.

14. The device according to claim 6, wherein at least one internal standard is selected from the group consisting of 7-aminoclonazepam-d4, Alprazolam-d5, Clonazepam-d4, Diazepam-d5, Flunitrazepam-d3, α-hydroxyalprazolam-d5, Lorazepam-d4, Midazolam-d4, Nitrazepam-d5, Nordiazepam-d5, Oxazepam-d5, Phenazepam-d4, Temazepam-d5, Amphetamine-d11, Benzoylecgonine-d3, Carisoprodol-d7 Cocaine-d3, Cyclobenzaprine-d3, MDA-d5, MDEA-d5, MDMA-d5, Methamphetamine-d5, Phencyclidine-d5, Phencyclidine-d5, Phentermine-d5, 6-MAM-d6, Acetyl fentanyl-d5, Acetyl norfentanyl-d5, Buprenorphine-d4, Codeine-d6, Dextromethorphan-d3, Dihydrocodeine-d6, EDDP-d3, Fentanyl-d5, Heroin-d9, Hydrocodone-d6, Hydromorphone-d3, Meperidine-d4, Meprobamate-d7, Methadone-d9, Morphine-d3, Naloxone-d5, Naltrexone-d3, Norbuprenorphine-d3, Norfentanyl-d5, Normeperidine-d4, Norpropoxyphene-d5, Oxycodone-d6, Propoxyphene-d5, Sufentenil-d5, Tramadol-13C-d3.

15. The device according to claim 1, wherein a plurality of the wells or the vials of the device further comprise one or a plurality of deconjugation enzymes.

16. The device according to claim 1, wherein the sample is urine, serum, plasma, blood, saliva, cerebral spinal fluid, feces, semen, or vaginal fluid.

17. The device according to claim 1, wherein the wells of the multi-well plate or the plurality of vials further comprise one or a plurality of tracers, wherein
a) the one or a plurality tracers are positioned in the wells or vials to allow for detection of cross-contamination between the wells or vials;
b) the one or a plurality of tracers can be detected independently of the internal standards; and
c) the one or a plurality of tracers are compatible with the assay and the instrument used to detect the analytes.

18. A kit for quantitative determination of the concentration of a plurality of analytes in one or a plurality of test samples comprising:
a) a device according to claim 1; and
b) a written description of the specifications of the device.

19. The kit according to claim 18, wherein the written description provides the precise amounts of the components in each well or vial of the device.

20. The kit according to claim 18, further comprising a solid phase extraction device.

21. The kit according to claim 20, wherein the solid phase extraction device is a supported liquid extraction plate or column.

22. The kit according to claim 18, further comprising standard operating procedures for measuring specific analytes in human urine or blood wherein the procedures are customized to meet specific end user validation requirements.
